(19) United States
(12) Patent Application Publication
(10) Pub. No.: US 2025/0235601 A1
(43) Pub. Date: Jul. 24, 2025

(54) MODULAR THERAPY DEVICES AND POWERED CONSUMABLES WITH DISPOSAL FEATURES FOR REDUCED ENVIRONMENTAL IMPACT

(71) Applicant: KCI Manufacturing Unlimited Company, Westmeath (IE)

(72) Inventor: Benjamin A. Pratt, Saint Paul, MN (US)

(21) Appl. No.: 18/705,394

(22) PCT Filed: Sep. 26, 2022
(86) PCT No.: PCT/IB2022/059118

§ 371 Date: Apr. 26, 2024

Related U.S. Application Data
(60) Provisional application No. 63/272,766, filed on Oct. 28, 2021.

(51) Int. Cl. A61M 1/00

(52) U.S. Cl. CPC A61M 1/98; A61M 2205/3344

(57) ABSTRACT

A modular therapy device for medical treatment includes a carrier, an electronic component fixedly coupled to the carrier, and a clinical body. The clinical body includes a fluid driver and a canister fluidly coupled to the clinical body. The canister defines an internal cavity configured to receive a volume of fluid therein. The clinical body is detachably coupled to the carrier. In some embodiments, the electronic component is one of a plurality of electronic components including a control board and a motor electrically coupled to the control board. The clinical body may include a flow manifold and the fluid driver may be disposed within the flow manifold. The fluid driver may be detachably coupled to the motor.

### CROSS-REFERENCE TO RELATED APPLICATIONS

This application is a U.S. National Stage Entry of PCT/IB2022/059118, filed Sep. 26, 2022, which claims the benefit, under 35 U.S.C. § 119 (e), of U.S. Provisional Application No. 63/272,766, filed on Oct. 28, 2021, which is incorporated herein by reference in its entirety.

### BACKGROUND

The present disclosure relates generally to therapy devices for medical treatment. More specifically, the present disclosure relates to modular therapy devices that are structured to separate clinical and electrical waste for effective disposal.

Many different types of therapy devices are available that interface with a patient to provide medical treatment. For example, negative pressure wound therapy (NPWT) is a therapeutic technique that includes using a therapy device to promote healing in acute or chronic tissue wounds. NPWT systems are configured to apply a negative pressure to a wound, through a dressing that is connected to the wound (e.g., to the skin surrounding the wound), to remove infectious materials, and to help promote wound closure and healing. The NPWT device may be include a pump and a canister that is fluidly connected to dressing and receives fluid from the wound. The NPWT device may also include fans, controllers, and other components that are positioned in the same enclosure as the pump. The various components of the NPWT device are generally enclosed in a single housing and are disposed of as a single unit, or in pieces that include a combination of electrical and non-electrical components, leading to considerable waste in the disposal process. It would be desirable to provide a medical therapy device that allows for separation of different materials and components to facilitate recycling and reuse, and that reduces the overall environmental impact associated with such devices.

### SUMMARY

One implementation of the present disclosure relates to a modular therapy device. The device includes a carrier, an electronic component fixedly coupled to the carrier, and a clinical body. The clinical body includes a fluid driver and a canister fluidly coupled to the clinical body. The canister defines an internal cavity configured to receive a volume of fluid therein. The clinical body is detachably coupled to the carrier.

In some embodiments, the electronic component is one of a plurality of electronic components including a control board and a motor electrically coupled to the control board. The clinical body may include a flow manifold and the fluid driver may be disposed within the flow manifold. The fluid driver may be detachably coupled to the motor.

In some embodiments, the carrier is slidably engaged with the clinical body. In some embodiments, the clinical body further includes a fluid driver subassembly including a flow manifold and a fluid driver disposed within the flow manifold. The canister may be detachably coupled to the fluid driver subassembly.

In some embodiments, the clinical body further includes a housing defining a first pressure chamber and a second pressure chamber that is fluidly isolated from the first pressure chamber. The fluid driver may be disposed within the first pressure chamber. The electronic component may include a pressure sensor that is detachably coupled to the second pressure chamber.

In some embodiments, the clinical body further includes a housing defining a flow manifold. The fluid driver may be disposed within the housing and the canister may be detachably coupled to the housing.

In some embodiments, the electronic component includes a motor and the device further includes a base subassembly having a control board that is coupled to the motor. The carrier may be detachably coupled to the base subassembly.

In some embodiments, the device further includes an expandable module that is detachably coupled to the base subassembly, the carrier, and the clinical body.

In some embodiments, the electronic component is a motor and the fluid driver is a pump configured to be detachably coupled to the motor.

In some embodiments, the clinical body further includes a plurality of fluid ports including a pressure sensing port configured to be fluidly coupled to a pressure sensor and a negative pressure port fluidly coupled to the canister.

In some embodiments, the carrier and the electrical component are disposable as waste electric and electronic equipment (WEEE) waste, and the clinical body is disposable as clinical waste independent from the WEEE waste.

Another implementation of the present disclosure relates to a negative pressure wound therapy device. The device includes a first housing subassembly having a plurality of electronic components that are disposable as WEEE waste. The device also includes a second housing subassembly having a pump and a canister fluidly coupled to the pump. The second housing subassembly is detachably coupled to the first housing subassembly and is disposable as clinical waste independent from the WEEE waste.

In some embodiments, the plurality of electronic components include a motor, a user interface, and a controller communicably coupled to the motor and the user interface. The motor may be structured to detachably couple to the pump by pressing the motor toward the pump.

In some embodiments, the plurality of electronic components includes a motor and a pressure sensor. The second housing may define a pressure chamber. The first housing subassembly may be slidably engaged with the second housing subassembly and may be configured such that sliding the first housing subassembly toward the second housing subassembly causes a shaft of the motor to engage with the pump and the pressure sensor to engage with the pressure chamber.

In some embodiments, the second housing subassembly includes a housing that is detachably coupled to the canister.

Yet another implementation of the present disclosure relates to a method of disposing a therapy device. The method includes separating a first housing subassembly from a second housing subassembly. The first housing subassembly includes a plurality of electronic components. The second housing subassembly includes a fluid driver and a canister fluidly coupled to the fluid driver. Separating the first housing subassembly from the second housing subassembly includes disengaging a motor of the first housing subassembly from the fluid driver of the second housing subassembly. The method also includes disposing at least a portion of the second housing subassembly as clinical waste.

In some embodiments, disposing at least the portion of the second housing subassembly includes separating the canister from a housing of the second housing subassembly by decoupling the canister from the fluid driver.

In some embodiments, separating the first housing subassembly from the second housing subassembly includes sliding a front housing of the first housing subassembly relative to a rear housing of the second housing subassembly.

In some embodiments, the method includes coupling a third housing subassembly to the first housing subassembly.

Yet another implementation of the present disclosure relates to a negative pressure wound therapy device. The device includes a carrier, a clinical body, and a flexible strap. The carrier includes a plurality of electrical components that are disposable as WEEE waste. The clinical body includes a canister sized to receive a volume of fluid therein. The flexible strap is coupled to and extends between the carrier and the clinical body. The flexible strap allows the clinical body to rotate toward and away from the carrier.

In some embodiments, the flexible strap includes a perforation extending along an intermediate position of the strap in between the canister and the clinical body. The perforation may be structured to allow a user to tear or snap apart the flexible strap at the intermediate position. In other embodiments, the flexible strap may include a fabric material.

In some embodiments, the device further includes a pump coupled to the carrier, a first fluid conduit fluidly coupling the pump to the canister, a pressure sensor coupled to the carrier, and a second fluid conduit coupled to the pressure sensor and extending around the clinical body.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE FIGURES

FIG. 1 is a side view of a negative pressure wound therapy (NPWT) device, according to an illustrative embodiment.

FIG. 2 is an exploded view of the NPWT device of FIG. 1.

FIG. 3 is a side view of a NPWT device, according to another illustrative embodiment.

FIG. 4 is a side view of a first housing subassembly of the NPWT of FIG. 3.

FIG. 5 is a side view of a second housing subassembly of the NPWT of FIG. 3.

FIG. 6 is a front perspective view of the first housing subassembly of FIG. 4.

FIG. 7 is a rear perspective view of the first housing subassembly of FIG. 4.

FIG. 8 is a front perspective view of the second housing subassembly of FIG. 4.

FIG. 9 is a rear perspective view of the second housing subassembly of FIG. 4.

FIG. 10 is a perspective view of a fluid manifold portion of the second housing subassembly of FIG. 4.

FIG. 11 is a rear perspective view of the NPWT device of FIG. 3.

FIG. 12 is a front view of an NPWT device, according to another illustrative embodiment.

FIG. 13 is a side view of an NPWT device, according to another illustrative embodiment.

FIG. 14 is an exploded view of the NPWT device of FIG. 13.

FIG. 15 is a side view of the NPWT device of FIG. 13.

FIG. 16 is a side view of a modular therapy device, according to an illustrative embodiment.

FIG. 17 is an exploded view of the modular therapy device of FIG. 16.

FIG. 18 is a side view of a modular therapy device, according to another illustrative embodiment.

FIG. 19 is an exploded view of the modular therapy device of FIG. 18.

### DETAILED DESCRIPTION

### Overview

Various countries and jurisdictions have implemented regulations for the disposal of waste electrical and electronic (WEEE) materials to reduce the environmental impact and to facilitate recovery of rare materials used in the production of some electronic components. Existing medical therapy devices generally include electronic components that are subject to WEEE regulations. However, these therapy devices also include fluid canisters, flow conduit, and other components that, due to their exposure to fluids and materials from a patient, must be disposed of as clinical waste. The therapy device may also include pumps, fan, and other components that blur the boundaries with respect to what constitutes clinical and electrical waste. For example, pumps may have been exposed to air from a wound, and fans may become exposed to water vapor from wound exudate during use. Moreover, separation of these components from an enclosure for appropriate disposal is often prohibitively difficult. As a result, much of the WEEE waste may need to be disposed of along with the clinical waste after each use.

Referring generally to the Figures, modular therapy devices are shown that provide a means of separating WEEE waste employed by the devices from clinical waste and/or other recyclable waste in a manner that reduces user impact, while increasing the chances that each of the device elements are disposed of in an environmentally friendly manner. In contrast to existing therapy devices, the modular devices of the present disclosure include a first housing subassembly that includes all of the WEEE components and a separate, second housing subassembly (e.g., clinical body, etc.) that includes all the non-WEEE components. In some embodiments, the second housing subassembly includes a fluid driver (e.g., pump, fan, etc.) that is detachable (e.g., removable by manual manipulation of the housing subassembly without destroying the first housing subassembly, etc.) from other electrical components and that can be disposed of at a landfill or incinerated with other clinical waste.

In at least one embodiment, the first housing subassembly and the second housing subassembly together form a NPWT device configured to apply a negative pressure to a wound and to receive fluid from the wound site (e.g., wound exudate, instillation fluids, etc.). The second housing subassembly may include the fluid canister. The second housing subassembly may be slidably engaged to the first housing subassembly such that the motor may be disengaged from the fluid driver by simply moving the second housing subassembly relative to the first housing subassembly, without requiring any additional user interaction (e.g., without requiring the user to manually disconnect electrical or fluid connections between the first housing subassembly and the second housing subassembly, etc.).

In at least one embodiment, the fluid driver and/or flow tubing may be detachably coupled from a fluid canister of the therapy device so as to allow the canister to be disposed of independent from the fluid driver and any plastic tubing without requiring separate tools and/or cutting operations. For example, the fluid driver and/or plastic tubing may be separated from the canister once the canister is filled with fluid. A second canister may then be secured to the fluid driver and/or plastic tubing in place of the first canister so that treatment can continue without requiring replacement of the entire second housing subassembly. At the conclusion of treatment (e.g., for a single patient), the fluid driver and/or plastic tubing may be recycled separately from the clinical waste, thereby reducing the overall environmental impact associated with disposal of the second housing subassembly. The first housing subassembly may be reused for other patients until the end of its useful life and then recycled as WEEE waste.

The modular therapy device may also be equipped with other powered consumables including, but not limited to, fans, supplemental pumps, oxygen generators, and/or other components that may be detachably coupled to the therapy device depending on the intended application.

### Modular Therapy Device

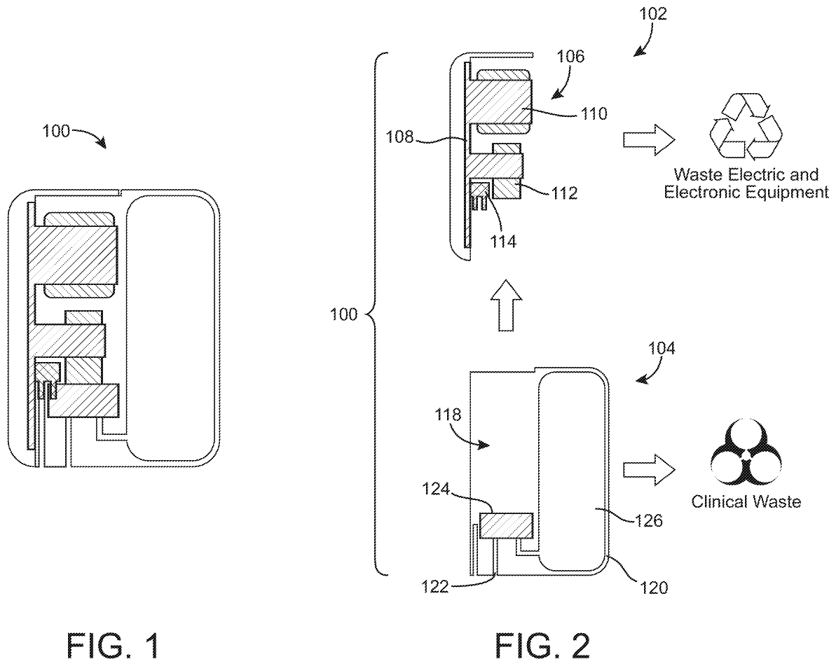

Referring to FIGS. 1 and 2, a modular therapy device 100 is shown, according to an illustrative embodiment. The modular therapy device 100 is configured as a NPWT device that is configured to apply a reduced pressure (e.g., negative pressure with respect to ambient) to a wound (e.g., a surgical wound, pressure wound, burn, skin graft site, or another type of wound or tissue injury). As shown in FIG. 1, the therapy device 100 is a self-contained (e.g., unitary, etc.) device that includes all of the components needed to administer and control negative pressure applied to a dressing. In some embodiments, the therapy device 100 may further include and/or be engageable with a track pad (e.g., a dressing applied to the patient's skin and configured to sealingly engage the therapy device 100 with the wound). For example, the therapy device 100 may include fluid ports and/or connections (e.g., push-to-connect fittings, etc.) that allow a user to attach the flow tubing to the therapy device 100 (e.g., the canister, pump, etc.).

As shown in FIG. 2, the therapy device 100 includes a first housing subassembly 102 and a second housing subassembly 104 that is detachably coupled to the first housing subassembly 102. The first housing subassembly 104 includes all of or a substantially majority of electronic components 106 for the therapy device 100 (e.g., the electronic components disposable as WEEE waste for the therapy device 100). In the embodiment of FIG. 1, the electronic components 106 include a controller 108 (e.g., control board(s), circuit board(s), control unit(s), etc.), a power supply 110, a motor 112, a pressure sensor 114, and any electrical connections connecting the electronic components 106 to the controller 108. The first housing subassembly also includes a front housing, show as carrier 116 (e.g., base, etc.) that is coupled to and supports the electrical components 106. In at least one embodiment, the entire first housing subassembly 102 may be disposed of as WEEE waste.

As shown in FIG. 2, the second housing subassembly 104 includes all of the components that come into contact or are otherwise exposed to fluids from the wound during device operations. As such, the second housing subassembly 104 may form a clinical body of the therapy device 100. The second housing subassembly 104 includes a rear housing 118 configured to form and/or support various components of the second housing subassembly 104. The second housing subassembly 104 also includes a canister 120, a flow manifold 122, and a fluid driver 124. The canister 120 is configured to be disposed along a flow path between the fluid driver 124 and the wound and to receive any fluids (e.g., wound exudate, instillation fluid, etc.) from the wound. The canister 120 defines an internal cavity 126 configured to receive and contain a volume of fluid therein.

The flow manifold 122 is configured to fluidly couple the various components of the therapy device 100 to one of the fluid driver 124 and/or pressure sensor 114. The flow manifold 122 includes a plurality of pneumatic pathways (e.g., fluid conduits, tubes, etc.). The flow manifold 122 may also include at least one pressure chamber sized to receive and support the fluid driver 124 therein. In the embodiment of FIG. 2, the canister 120, the flow manifold 122, and the pressure chamber are a single unitary body that cannot be separated without breaking apart or damaging the second housing subassembly 104. For example, the canister 120 and/or flow manifold 122 may be at least partially integrally formed with the rear housing 118 (e.g., molded from a single piece of plastic, etc.). In other embodiments, parts of the canister 120 and/or flow manifold 122 may be welded (e.g., ultrasonically) or otherwise fixedly coupled to the rear housing 118.

As shown in FIG. 2, the second housing subassembly 104 is detachably (e.g., removably, etc.) coupled to the first housing subassembly 104 such that the second housing subassembly 104 can be separated from the first housing subassembly 104 without breaking apart pieces of the first or second housing subassemblies. In the embodiment of FIG. 2, the second housing subassembly 104 may be pulled apart from the first housing subassembly 102 (e.g., downward from the first housing subassembly 104 in FIG. 2) to disengage (i) the fluid driver 124 from the motor 112 and (ii) the flow manifold 122 from the pressure sensor 114. The second housing subassembly 104 may include sealing members (e.g., grommets, gaskets, etc.) that form a press-fit with the pressure sensor to ensure a seal is maintained between the pressure sensor and the pressure chamber when the first housing subassembly 102 is coupled to the second housing subassembly 102 and to allow separation of the pressure sensor without requiring the user to interact with any fluid connectors and/or fittings. In some embodiments, the first housing subassembly 104 may be reusable. For example, upon filling the canister 120, a clinician and/or other user may replace the second housing subassembly 104 with a third housing subassembly that includes an empty canister.

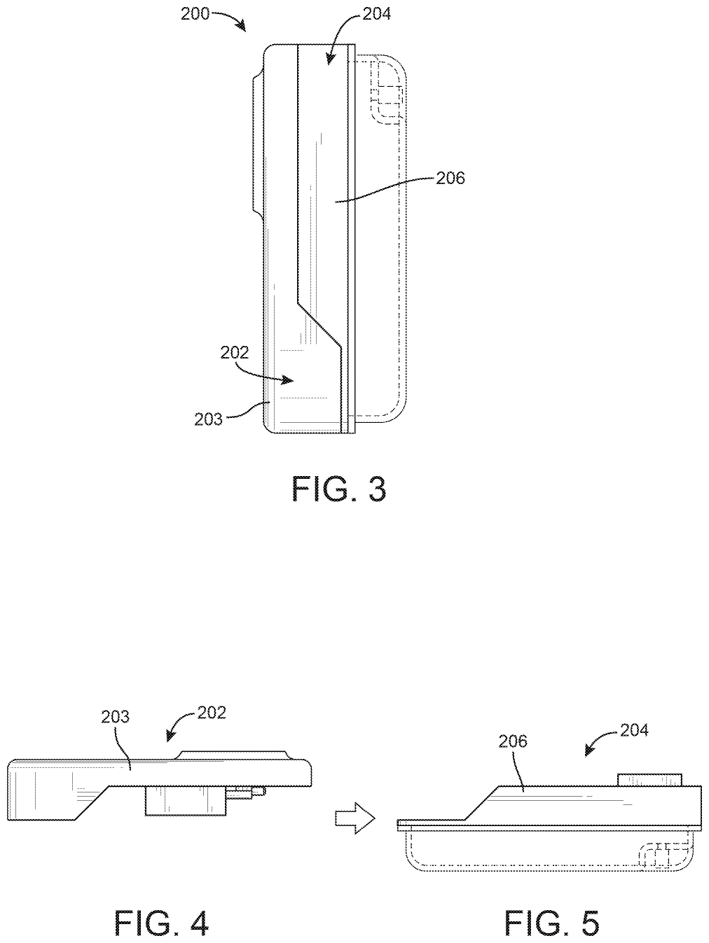

Referring now to FIG. 3, another modular NPWT therapy device 200 is shown, according to an illustrative embodiment. The therapy device 200 may be the same as or similar to the therapy device 100 of FIGS. 1 and 2. The therapy device 200 includes a first housing subassembly 202 including a carrier 203 (e.g., front housing, etc.) and a second housing subassembly 204 (e.g., clinical body, etc.) that is detachably coupled to the carrier 203. In particular, the second housing subassembly 204 includes a rear housing 206 that is slidably engaged with the carrier 203. The carrier 203 may include an outer shell and a pair of tracks (e.g., recessed areas, slots, etc.) coupled to the outer shell. The rear housing 206 may include protrusions (e.g., a guide rib, etc.) on either end of the rear housing 206 that are engageable with the tracks to couple the second housing subassembly 204 to the first housing subassembly 202. In other embodiments, the therapy device 200 may include a latch, clips, screws, and/or another suitable mechanical fastener to detachably couple the rear housing 206 to the carrier 203. Beneficially, the sliding interface between the rear housing 206 and the carrier 203 reduces the amount of user interaction required to separate and dispose of the second housing subassembly 204 when compared to using conventional mechanical fasteners. However, it will be appreciated that any other detachable coupling may be used to secure the first housing subassembly to the second housing subassembly 204 without departing from the inventive principles disclosed herein.

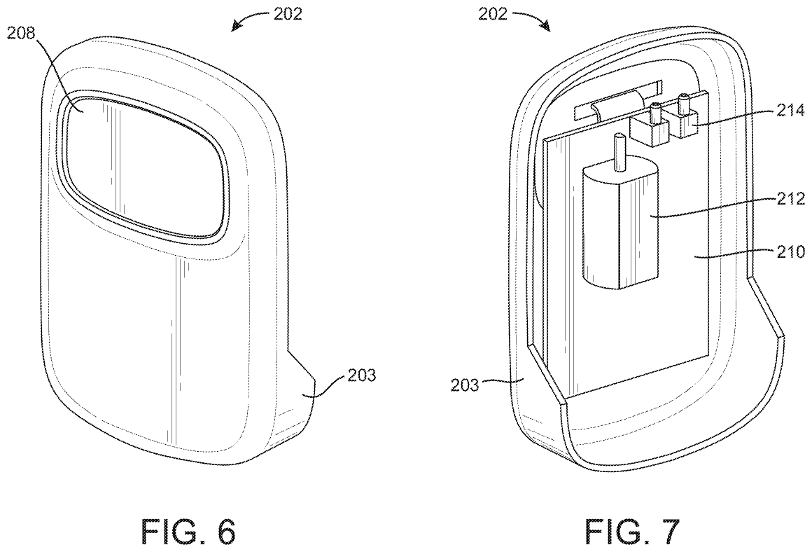

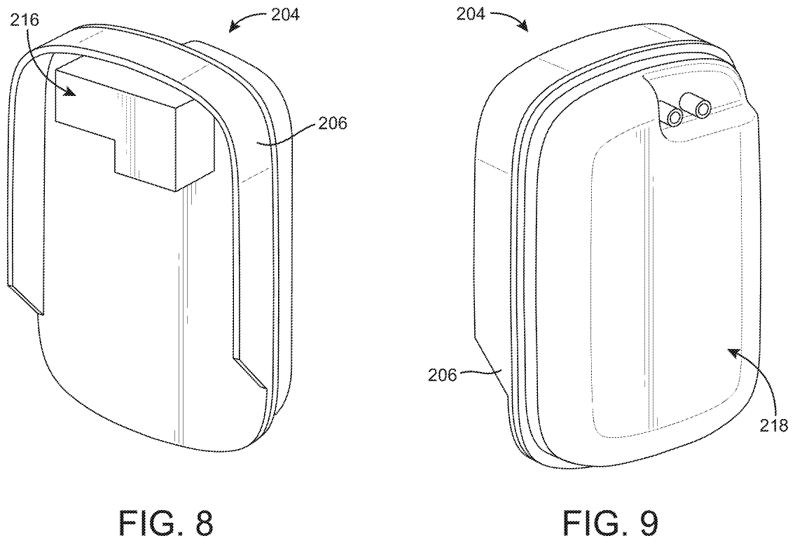

FIGS. 6 and 7 show front and rear views, respectively of the first housing subassembly 202 independent from the second housing subassembly 204. As shown in FIGS. 6 and 7, the first housing subassembly 202 includes a plurality of electrical components that are disposable as WEEE waste. In particular, the first housing subassembly includes a user interface 208, controller 210, a motor 212, and at least one pressure sensor 214. In other embodiments, the front housing subassembly may include additional, fewer, and/or different components.

As shown in FIG. 6, the user interface 208 is coupled to the carrier 203 (e.g., front housing) and is configured to display operating parameters and facilitate operator interaction with the therapy device 200. The user interface 208 may include a membrane (e.g., a thin transparent label, etc.) and light emitting diodes configured to shine through the membrane to notify the user of the operating status of the therapy device 200 and/or measured parameters (e.g., pressure at the wound, pump pressure, treatment duration, fluid levels in the canister, etc.). In other embodiments, the user interface 208 may include a touchscreen and/or another suitable input/output device.

As shown in FIG. 7, the controller 210, the motor 212, and at least one pressure sensor 214 are disposed within a recessed area of the carrier 203. The controller 210 includes a printed circuit board (PCB) that is coupled to the carrier 203 and that supports the motor 212 and the pressure sensor(s) 214. In at least one embodiment, the controller includes a processing circuit, including a processor and memory. The processing circuit is communicably coupled to the motor 212, pressure sensors 214, and user interface 208 and is configured to coordinate communications between (and operation of) the motor 212, pressure sensors 214, and the user interface 208. For example, the processing circuit may be configured to control operation of the motor 212 in response to pressure readings from the pressure sensor(s) 214 (e.g., a measured pressure at the wound, etc.).

The motor 212 is configured to power a fluid driver in the second housing subassembly 204. As shown in FIG. 7, the motor 212 includes a driveshaft, which may include a mechanical coupling to facilitate engagement with the fluid driver and to prevent relative rotation between the fluid driver and the driveshaft when the second housing subassembly 204 (see FIG. 3) is coupled to the carrier 203. The pressure sensors 214 are configured to measure a pneumatic pressure at one of the wound and the fluid driver. In the embodiment of FIG. 7, the first housing subassembly includes two pressure sensors 214 including a first pressure sensor configured to monitor a pressure at the wound and a second pressure sensor configured to monitor an operating pressure of the fluid driver. In some embodiments, the first housing subassembly may also include a power source for the therapy device 200.

FIGS. 8 and 9 show front and rear views, respectively, of the second housing subassembly 204 (e.g., clinical body) of the therapy device 200. As shown, the second housing subassembly 204 includes a rear housing 206 that is configured to slidably engage with the carrier 203 to detachably couple the rear housing 206 to the carrier 203. The second housing subassembly 204 also includes a flow manifold 216 and a canister 218. The flow manifold 216 is coupled to a forward end of the rear housing 206 and disposed in a recessed area of the rear housing 206. The canister 218 is coupled to a rear end of the rear housing 206 and extends along an entire length of the rear housing 206. It will be appreciated that the arrangement and/or geometry of the flow manifold 216 and the canister 218 may be different in other embodiments. In other embodiments, the second housing subassembly 204 may include additional, fewer, and/or different components.

As shown in FIGS. 8 and 9, the canister 218 extends away from the rear end of the rear housing 206. The rear housing 206 and the canister 218 together form an internal cavity that is configured to receive a volume of fluid (e.g., wound exudate, instillation fluid, etc.) therein. In some embodiments, the canister 218 may be sized to receive an absorbent and/or superabsorbent material (e.g., an absorbent pad, an absorbent layer, etc.). In the embodiment of FIGS. 8 and 9, the canister 218 is formed from a transparent plastic material, which provides visual access to a clinician or another user to identify an amount of fluid that has been drawn away from the wound.

Returning to FIG. 8, the flow manifold 216 is disposed in a recessed area of the rear housing 206. The flow manifold 216 is structured to house the fluid driver, facilitate application of negative pressure to the wound, and fluidly couple the pressure sensor to the wound to facilitate monitoring and control of the therapy device. As shown in FIG. 8, the flow manifold 216 extends away from a forward surface of the rear housing 206 in a substantially perpendicular orientation relative to the forward surface.

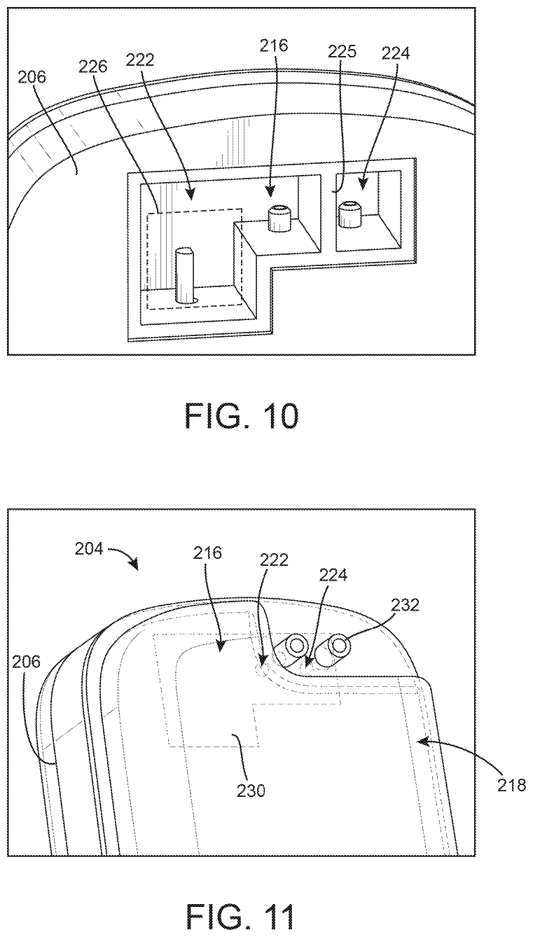

Referring to FIG. 10, a perspective view of the flow manifold 216 is shown, according to an illustrative embodiment. In at least one embodiment, the flow manifold 216 is integrally formed with the rear housing 206 from a single piece of material (e.g., injection molded from plastic, etc.). In other embodiments, the flow manifold 216 is formed separately from the rear housing 206 and welded (e.g., ultrasonically, etc.) or otherwise coupled to the rear housing 206. As shown in FIG. 10, the flow manifold 216 includes a plurality of connectors 220 (e.g., ports, etc.) structured to interface with (e.g., engage with, etc.) the motor and pressure sensors of the first housing subassembly. In at least one embodiment, the connectors 220 include openings in an outer wall of the flow manifold 216 and sealing members (e.g., gaskets, O-rings, grommets, etc.) disposed in the openings that are configured to sealingly engage one of (i) the driveshaft of the motor, or (ii) pressure sensors to the flow manifold 216. In the embodiment of FIG. 10, the flow manifold 216 includes two pneumatic chambers, shown as first pressure chamber 222 and second pressure chamber 224. The flow manifold 216 also includes a partition 225 (e.g., wall, etc.) that fluidly isolates the first pressure chamber 222 from the second pressure chamber 224. It will be appreciated that the number and/or arrangement of chambers may be different in other embodiments.

As shown in FIG. 10, the first pressure chamber 222 is sized to receive the fluid driver 226 therein. The fluid driver 226 may be a pump (e.g., a pump head of a diaphragm pump, etc.) configured to apply reduced pressure to the first pressure chamber 222. In other embodiments, the fluid driver 226 may be another form of fluid delivery device (e.g., a fan, etc.). The flow manifold 216 may also include supports (e.g., clips or another suitable mechanical fastener) that are engageable with the fluid driver 226 to couple the fluid driver 226 to the flow manifold 216. The first pressure chamber 222 also includes two connectors 220, including a first connector adapted to receive and sealingly engage with the driveshaft of the motor, and a second connector adapted to receive and sealingly engage with one of the pressure sensors (e.g., a first pressure sensor). In this way, the negative pressure provided by the fluid driver (e.g., pump) can be monitored by the pressure sensor during operation. The first pressure chamber 22 may also include a fluid discharge port (e.g., connection, conduit, etc.) coupled to the fluid driver through which the fluid driver can exhaust any air received through the canister 218.

As shown in FIG. 10, the second pressure chamber 224 is separated from the first pressure chamber 222 by a partition 225 (e.g., wall, barrier, etc.), and includes a connector 220 that is adapted to receive and sealingly engage with one of the pressure sensors (e.g., a second pressure sensor). The second pressure chamber 224 may be a wound pressure monitoring chamber that is configured to be fluidly coupled to a wound so that the pressure at the wound can be monitored during treatment.

As shown in FIG. 11, the second housing subassembly 204 additionally includes a cover 230 coupled to the rear housing 206 and/or flow manifold 216 that encloses the first pressure chamber 222 and the second pressure chamber 224 and separates the flow manifold 216 from the canister 218. The cover 230 may include a plate and/or label that is glued, ultrasonically welded, or otherwise coupled to the rear housing 206 and/or flow manifold 216. As shown in FIG. 11, the cover 230 and canister 218 include fluid ports 232 (e.g., connections, etc.) for each of the first pressure chamber 222 and the second pressure chamber 224, and that may be used to couple a respective one of the chambers to the canister 218 or wound. In some embodiments, the second housing subassembly 204 may also include at least one filter element between the pump and the canister 218 and/or between the second pressure chamber 224 and the wound to prevent fluid ingestion into the flow manifold. In the embodiment of FIGS. 10 and 11, the filter element may include a low cost filter media and/or fluid separator. In other embodiments (e.g., embodiments in which the canister 218 is separable from the rear housing 206), the filter element may include an antimicrobial filter element to allow the rear housing 206 and flow manifold 216 to be reused multiple times.

A method of disposing a therapy device (e.g., the NPWT device 200 of FIG. 3) may include separating the first housing subassembly 202 from the second housing subassembly 204 by, for example, sliding the carrier 203 with respect to the rear housing 206 (e.g., sliding the carrier 203 away from the rear housing 206). In some embodiments, the method may further include separating the canister 218 from the rear housing 206. The method may also include decoupling the canister 218 from the fluid driver 226 so that portions of the second housing subassembly 204 may be reused. The method may further include coupling a third housing subassembly to the first housing subassembly 202 (which may be the same as or similar to the second housing subassembly 204) so that the first housing subassembly can be reused. For example, the method may include attaching a new canister and/or rear housing to the first housing subassembly 202 after the old canister 218 has been filled with fluid. The method may include disposing of the second housing subassembly as clinical waste (and/or the rear housing separately from the canister as recyclable waste). In other embodiments, the method may include additional, fewer, and/or different operations.

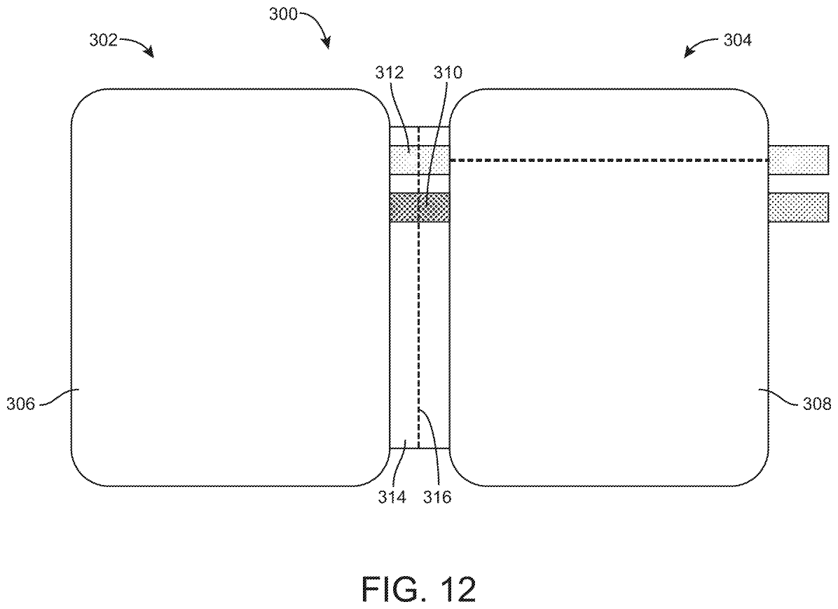

It will be appreciated that various modifications to the design and arrangement of components for the NPWT therapy device may be made without departing from the inventive concepts described herein. For example, referring to FIG. 12, another NPWT therapy device 300 is shown, according to an illustrative embodiment. The therapy device 300 includes a flexible coupling between the first housing subassembly 302 (e.g., a carrier 306 of the first housing subassembly 302) and a second housing subassembly 304 that allows movement of the first housing subassembly 302 with respect to the second housing subassembly 304. The second housing subassembly 304 may form a clinical body of the therapy device 300 that is disposable as clinical waste. In some embodiments, the second housing subassembly 304 may include a bag with a superabsorbent material. In other embodiments, the second housing subassembly 304 may include a rigid canister housing a superabsorbent material. In the embodiment of FIG. 12, the first housing subassembly 302 includes a plurality of electronic components that are disposable as WEEE waste. Unlike the NPWT therapy device 200 of FIGS. 3-11, the first housing subassembly 302 of FIG. 12 also includes a pump coupled to the carrier 306. The other electronic components of the therapy device 300 are also coupled to the carrier 306 along with fluid conduits, shown as pump conduit 310 and wound conduit 312, which are configured to fluidly couple the pump to a canister 308 of the second housing subassembly 304 and to monitor the pressure at the wound, respectively.

As shown in FIG. 12, the therapy device 300 also includes a flexible strap 314 coupled to and extending between the first housing subassembly 302 (e.g., carrier 306) and the second housing subassembly 304 (e.g., canister 308). The flexible strap 314 is structured to allow the second housing subassembly 304 to rotate toward and away from the first housing subassembly 302. Beneficially, the flexible strap 314 provides freedom of movement to facilitate application (e.g., mounting or arrangement) of the therapy device 300 to a patient. In some embodiments, the second housing subassembly 304 may be rotated into contact with a rear surface of the first housing subassembly 302 and clipped or otherwise fastened to the first housing subassembly 302 to make the overall therapy device 300 more compact. The flexible strap 314 may include a fabric material, rubber, and/or another suitably non-rigid material that allows movement of the first and second housing subassemblies with respect to one another.

In the embodiment of FIG. 12, the second housing subassembly 304 is separable from the first housing subassembly 302 by tearing, ripping, and/or cutting apart the flexible strap 314. For example, as shown in FIG. 12, the flexible strap 314 may include a perforation 316 extending along an intermediate position (e.g., central position halfway between the first and second housing subassemblies, etc.) between the first and second housing subassemblies. The perforation 316 may be a plurality of small cuts spaced at intervals along a reference line that extends between lateral ends of the flexible strap 314 (e.g., in a vertical direction as shown in FIG. 12). The perforation 316 may be structured to facilitate tearing of the flexible strap 314. In other embodiments, the first and/or second housing subassemblies may be detachably coupled to the flexible strap 314, or the flexible strap 314 may include buttons, zippers, and/or another suitable fastener to disconnect the first housing subassembly 302 from the second housing subassembly 304. As shown in FIG. 12, the wound conduit 312 may also include a perforation (e.g., along an outer radius of the conduit) that allows a user to snap off the tube after use (e.g., to snap off a portion of the wound conduit 312 that has been exposed to air from the wound). The pump conduit 310 may be detachably coupled to the first housing subassembly 302 and/or the second housing subassembly 304 to allow separation of the first and second housing subassemblies after use.

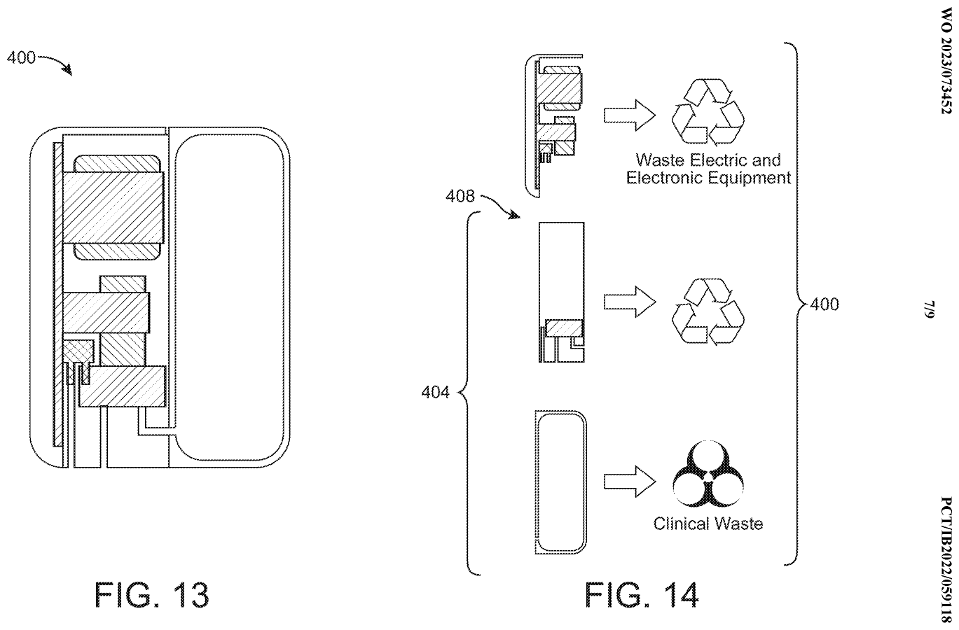

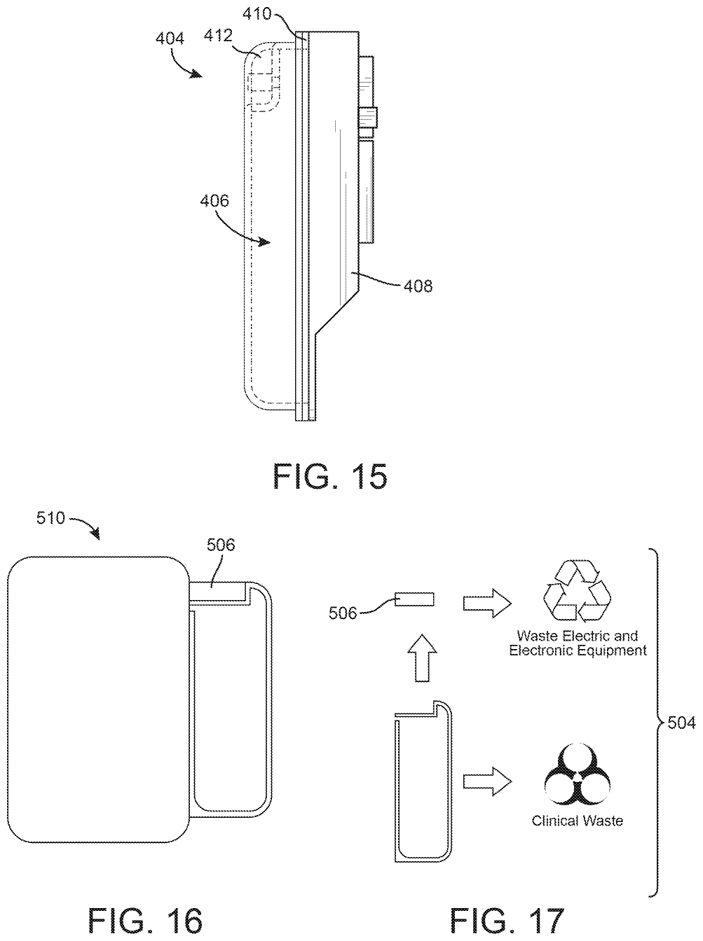

Referring to FIGS. 13 and 14, a therapy device 400 is shown in which a housing portion, shown as housing 408, of the second housing subassembly 404 is removable from the canister 406. The housing 408 may be the same as or similar to the rear housing 206 of the therapy device 200 described with reference to FIGS. 3-11. However, unlike the embodiment in FIGS. 3-11, the canister 406 of the second housing subassembly 404 may be a self-contained unit that is detachably coupled to the housing 408 so that the canister 406 can be separated from the housing 408 and/or the flow manifold. In this way, any plastics and/or other materials forming the housing 408 and flow manifold may be recycled instead of having to dispose of the housing 408 along with the canister 406 as clinical waste. FIG. 15 shows a side view of the second housing subassembly 404. The second housing subassembly 404 is shown to include a canister lid 410 and a canister body 412 coupled to the lid 410 that together form an internal cavity. The canister 406 (e.g., canister lid 410) may be coupled to the housing 408 using clips, latches, and/or another suitable mechanical fastener.

Beneficially, the housing 408 of the therapy device 400 of FIGS. 13-15 may be reused multiple times (e.g., with multiple different canisters 406). For example, the therapy device 400 may include filter elements that prevent fluid ingestion into the housing 408 (e.g., flow manifold, pump, etc.) and allow the housing 408 to be reused multiple times for a single patient. In some embodiments, the filter elements may include an antimicrobial filter to allow the housing 408 to be reused across multiple patients. The housing 408 may be made from recyclable plastics or another suitable material. After use, the housing 408 may be recycled separately from the clinical waste (e.g., separately from the canister 406) and the WEEE waste (the first housing subassembly 402) to further reduce the environmental footprint of the therapy device 400.

Referring to FIGS. 16 and 17, a side view and partially exploded view, respectively, of a modular therapy device 500 is shown, according to an illustrative embodiment. The therapy device 500 may be an NPWT device or another medical therapy device for diagnostics and/or treatment. For example, the device 500 may be similar to the NPWT device 200 of FIGS. 3-11 but may further include a powered consumable 506 that is coupled to the second housing subassembly 504 (e.g., canister subassembly, etc.). The powered consumable 506 may be a sensor (e.g., an exudate level sensor configured to monitor a level of fluid within the canister, pressure sensor configured to monitor a pressure in the canister and/or wound, etc.), a fluid driver (e.g., fan, pump, etc.), and/or another powered and/or electronic device. For example, the fluid driver may include a fan that is coupled to the canister to evaporate water content out of the wound exudate within the canister to prolong canister life. The powered consumable 506 may be decoupled from the canister and/or first housing subassembly and may be disposed of separately from the canister and/or first housing subassembly as WEEE waste. The first housing subassembly may then be reused as many times as desired or until failure of the components contained within the first housing subassembly (e.g., the first housing subassembly may be reused over periods of years, the powered consumable 506 may be reused for periods of weeks, and the canister may be replaced after each use, once a day or every other day, etc.).

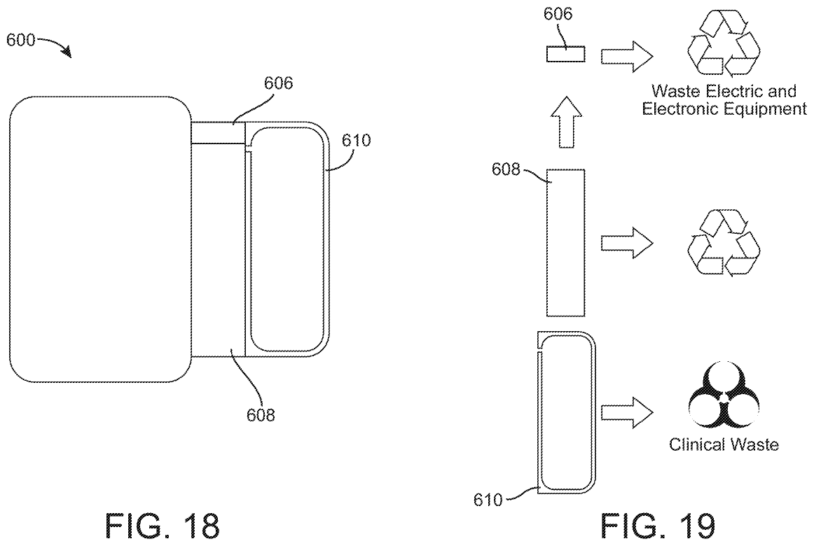

Referring to FIGS. 18 and 19, another example embodiment of a modular therapy device 600 is shown. The therapy device 600 may be similar to the therapy device 500 of FIGS. 16 and 17, but further includes a powered module 608 that is detachably coupled to a base therapy device 602 (e.g., a first housing subassembly including a controller, connectors, power source, base subassembly, etc.). The powered module 608 may also be detachably coupled to other components of the therapy device 600 including a carrier of the first housing subassembly, and the canister (e.g., the clinical body, etc.). The powered module 608 may include an additional pump for the therapy device 600 (e.g., a second pump to complement a first pump of the base therapy device 602) to improve pneumatic performance, an oxygen generating module, and/or another form of powered and/or electronic device. In this way, the first housing subassembly may be modified as needed to suit the needs of the particular therapy (or may be used for different therapies depending on the number and arrangement of modular components). As shown in FIG. 19, each component and/or module of the therapy device 600 may be separated for disposal purposes and to reduce the overall environmental impact after use. For example, the first housing subassembly may be reused as many times as desired or until component failure. The powered consumable 606 may be recycled as WEEE waste. The powered module 608 may be disposed of in a landfill or otherwise recycled. The canister 610 may be disposed of as clinical waste (e.g., incinerated, etc.). It will be appreciated that the number and/or arrangement of the powered modules 608 and powered consumables 606 may be different in other embodiments.

### Configuration of Exemplary Embodiments

The construction and arrangement of the systems and methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions and arrangement of the exemplary embodiments without departing from the scope of the present disclosure.

1. A modular therapy device, comprising:
a carrier;
an electronic component fixedly coupled to the carrier; and
a clinical body comprising a fluid driver and a canister fluidly coupled to the clinical body, the canister defining an internal cavity configured to receive a volume of fluid therein, the clinical body detachably coupled to the carrier.

2. The modular therapy device of claim 1, wherein the electronic component is one of a plurality of electronic components including a control board and a motor electrically coupled to the control board, the clinical body comprising a flow manifold and the fluid driver disposed within the flow manifold, the fluid driver detachably coupled to the motor.

3. The modular therapy device of claim 1, wherein the carrier is slidably engaged with the clinical body.

4. The modular therapy device of claim 1, wherein the clinical body further comprises a fluid driver subassembly including a flow manifold and the fluid driver disposed within the flow manifold, wherein the canister is detachably coupled to the fluid driver subassembly.

5. The modular therapy device of claim 1, wherein the clinical body further comprises a housing defining a first pressure chamber and a second pressure chamber that is fluidly isolated from the first pressure chamber, wherein the fluid driver is disposed within the first pressure chamber, the electronic component comprising a pressure sensor that is detachably coupled to the second pressure chamber.

6. The modular therapy device of claim 1, wherein the clinical body further comprises a housing defining a flow manifold, wherein the fluid driver disposed within the housing, and wherein the canister is detachably coupled to the housing.

7. The modular therapy device of claim 1, wherein the electronic component includes motor, wherein the modular therapy device further comprises a base subassembly having a control board that is coupled to the motor, and wherein the carrier is detachably coupled to the base subassembly.

8. The modular therapy device of claim 7, further comprising an expandable module detachably coupled to the base subassembly, the carrier, and the clinical body.

9. The modular therapy device of claim 1, wherein the electronic component is a motor and wherein the fluid driver is a pump configured to be detachably coupled to the motor.

10. The modular therapy device of claim 1, wherein the clinical body further comprises a plurality of fluid ports including a pressure sensing port configured to be fluidly coupled to a pressure sensor and a negative pressure port fluidly coupled to the canister.

11. The modular therapy device of claim 1, wherein the carrier and electrical component are disposable as waste electric and electronic equipment (WEEE) waste, and the clinical body is disposable as clinical waste independent from the WEEE waste.

12. A negative pressure wound therapy device, comprising:
a first housing subassembly comprising a plurality of electronic components that are disposable as WEEE waste; and
a second housing subassembly comprising a pump and a canister fluidly coupled to the pump, the second housing subassembly detachably coupled to the first housing subassembly and disposable as clinical waste independent from the WEEE waste.

13. The negative pressure wound therapy device of claim 12, wherein the plurality of electronic components comprises a motor, a user interface, and a controller communicably coupled to the motor and the user interface, and wherein the motor is structured to detachably couple to the pump by pressing the motor toward the pump.

14. The negative pressure wound therapy device of claim 12, wherein the plurality of electronic components comprises a motor and a pressure sensor, wherein the second housing defines a pressure chamber, wherein the first housing subassembly is slidably engaged with the second housing subassembly such that sliding the first housing subassembly toward the second housing subassembly causes a shaft of the motor to engage with the pump and the pressure sensor to engage with the pressure chamber.

15. The negative pressure wound therapy device of claim 12, wherein the second housing subassembly includes a housing defining a first pressure chamber and a second pressure chamber that is fluidly isolated from the first pressure chamber, wherein the pump is disposed in the first pressure chamber.

16. The negative pressure wound therapy device of claim 12, wherein the second housing subassembly comprises a housing detachably coupled to the canister.

17.-20. (canceled)

21. A negative pressure wound therapy device, comprising:
a carrier including a plurality of electrical components that are disposable as WEEE waste;
a clinical body comprising a canister sized to receive a volume of fluid therein; and
a flexible strap coupled to and extending between the carrier and the clinical body, the flexible strap allowing the clinical body to rotate toward and away from the carrier.

22. The negative pressure wound therapy device of claim 21, wherein the flexible strap includes a perforation extending along an intermediate position of the flexible strap between the canister and the clinical body.

23. The negative pressure wound therapy device of claim 22, wherein the perforation is structured to allow a user to tear or snap apart the flexible strap at the intermediate position.

24. The negative pressure wound therapy device of claim 21, wherein the flexible strap includes a fabric material.

25. The negative pressure wound therapy device of claim 21, further comprising:
a pump coupled to the carrier;
a first fluid conduit fluidly coupling the pump and the canister;
a pressure sensor coupled to the carrier; and
a second fluid conduit coupled to the pressure sensor and extending around the clinical body.
